(19) United States
(12) Patent Application Publication
(10) Pub. No.: US 2021/0283198 A1
(43) Pub. Date: Sep. 16, 2021

(54) METHOD FOR TREATING AND/OR PREVENTING COCCIDIOSIS

(71) Applicant: Life Rainbow Biotech Co., Ltd., Yilan County (TW)

(72) Inventors: Yu-Hsiang Yu, Yilan County (TW); Yeong-Hsiang Cheng, Yilan County (TW); Chia-Min Wu, Yilan County (TW); Rou-Wan Liao, Yilan County (TW); Ching-Kuo Yang, Yilan County (TW)

(21) Appl. No.: 16/816,903

(22) Filed: Mar. 12, 2020

(51) Int. Cl. A61K 35/742; A61P 33/02; A61K 9/00; A23K 50/75; A23K 10/12; A23K 10/18; A23K 20/147; A23K 20/195; A23K 20/158

(52) U.S. Cl. CPC A61K 35/742; A61P 33/02; A61K 9/0053; A23K 50/75; A23K 20/158; A23K 10/18; A23K 20/147; A23K 20/195; A23K 10/12

(57) ABSTRACT

The present disclosure provides a method for treating and/or preventing coccidiosis, including administering to a subject in need thereof a composition including an effective amount of a Bacillus spp.-fermented product. The Bacillus spp.-fermented product has the potential for development as feed additives and use as a possible solution to treat and/or prevent coccidiosis in the poultry industry.

### BACKGROUND OF THE INVENTION

### 1. Field of the Invention

The present invention relates to a method for treating and/or preventing coccidiosis, comprising administering to a subject in need thereof a composition comprising an effective amount of a Bacillus spp.-fermented product.

### 2. The Prior Art

Coccidiosis is the most important enteric diseases in poultry caused mainly by parasites from the Eimeria genus. Coccidiosis costs the poultry industry about 3 billion US dollars annually worldwide due to high mortality, impaired growth and high medical costs (Lillehoj H S, Lillehoj E P. 2006. Avian coccidiosis. A review of acquired intestinal immunity and vaccination strategies. Avian Dis. 44:408-425). Anti-coccidial drugs have been used to control coccidiosis, but drug resistance of Eimeria in chickens has become prevalent worldwide. Since the disadvantages of current anti-coccidial drugs and vaccines (Dalloul R A, Lillehoj H S. 2006. Poultry coccidiosis: Recent advancements in control measures and vaccine development. Expert Rev. Vaccines 5:143-163), alternative strategies to prevent coccidiosis in broilers are an urgent unmet need in the poultry industry.

Over the past decades, dietary supplementation of probiotics in broilers could provide similar benefits as antibiotics in terms of growth performance and disease prevention. Bacillus licheniformis, a gram-positive and endospore-forming probiotic strain, was identified from the gastrointestinal tract of broilers. The surfactin, B. licheniformis-derived cyclic lipopeptide, is an important antimicrobial peptide with antibacterial activity through disruption of the bacterial membrane (Carrillo C, Teruel J A, Aranda F J, Ortiz A. 2003. Molecular mechanism of membrane permeabilization by the peptide antibiotic surfactin. Biochim Biophys. Acta. 1611:91-97; Thaniyavarn J, Roongsawang N, Kameyama T, Haruki M, Imanaka T, Morikawa M. Kanaya S. 2003. Production and characterization of biosurfactants from Bacillus licheniformis F2.2. Biosci. Biotechnol. Biochem. 67:1239-1244; Lin E R, Cheng Y H, Hsiao F S H, Proskura W S, Dybus A, Yu Y H. 2019. Optimization of solid-state fermentation conditions of Bacillus licheniformis and its effects on Clostridium perfringens-induced necrotic enteritis in broilers. R. Bras. Zootec. 48:e20170298. doi:10.1590/rbz4820170298; Horng Y B, Yu Y H, Dybus A, Hsiao F S H, Cheng Y H. 2019. Antibacterial activity of Bacillus species-derived surfactin on Brachyspira hyodysenteriae and Clostridium perfringens. AMB Express 9:188. doi: 10.1186/s13568-019-0914-2). Supplementation of B. licheniformis in drinking water or in the diet improved the growth performance of broilers. Furthermore, B. licheniformis is able to improve growth performance and alleviate Clostridium perfringens-induced necrotic enteritis in broilers. Limited information is available on the beneficial effects of B. licheniformis as probiotics in broilers infected with coccidian parasites. However, the underlying mechanisms of how B. licheniformis reduces coccidiosis in broilers remain to be further investigated.

In the present invention, the applicant investigated the anti-coccidial efficacy of surfactin from B. licheniformis-fermented products on Eimeria tenella and the effects of B. licheniformis-fermented products in broilers exposed to Eimeria tenella-induced coccidiosis. The results provide valuable insights for the use of B. licheniformis-fermented products as a possible method for preventing coccidia in the poultry industry.

### SUMMARY OF THE INVENTION

A primary objective of the present invention is to provide a method for treating and/or preventing coccidiosis, comprising administering to a subject in need thereof a composition comprising an effective amount of a Bacillus spp.-fermented product.

According to an embodiment of the present invention, the Bacillus spp. is Bacillus licheniformis.

According to an embodiment of the present invention, the effective amount of the Bacillus licheniformis-fermented product is at least 1.25 g/kg.

According to an embodiment of the present invention, the coccidiosis is induced by Eimeria spp.

According to an embodiment of the present invention, the coccidiosis is induced by Eimeria tenella.

According to an embodiment of the present invention, the coccidiosis is treated and/or prevented by inhibiting sporulation of oocysts from the Eimeria tenella.

According to an embodiment of the present invention, the coccidiosis is treated and/or prevented by promoting death of merozoite from the Eimeria tenella.

According to an embodiment of the present invention, the coccidiosis is treated and/or prevented by disrupting morphology of sporozoites from the Eimeria tenella.

According to an embodiment of the present invention, the subject in need thereof is a poultry.

According to an embodiment of the present invention, the poultry is a broiler chicken.

According to an embodiment of the present invention, the coccidiosis is treated and/or prevented by alleviating cecal lesion and bloody diarrhea in the broiler chicken.

According to an embodiment of the present invention, the Bacillus licheniformis-fermented product comprises at least one antibacterial cyclic lipopeptide.

According to an embodiment of the present invention, the at least one antibacterial cyclic lipopeptide is surfactin.

According to an embodiment of the present invention, the surfactin is at least in an amount of 5 ppm.

According to an embodiment of the present invention, the composition further comprises a pharmaceutically acceptable carrier.

According to an embodiment of the present invention, the composition is in a form of powder, granule, liquid, gel or paste.

According to an embodiment of the present invention, the composition is prepared in a form of a medicament or a food product.

According to an embodiment of the present invention, the food product is a feed additive.

According to an embodiment of the present invention, the medicament is in a dosage form for oral administration.

According to an embodiment of the present invention, the dosage form is selected from the group consisting of solution, suspension, and powder.

In summary, the composition comprising an effective amount of a Bacillus licheniformis-fermented product provides the effect on treating and/or preventing coccidiosis through inhibiting sporulation of oocysts from the Eimeria tenella, promoting death of merozoite from the Eimeria tenella, disrupting morphology of sporozoites from the Eimeria tenella, and alleviating cecal lesion and bloody diarrhea in the broiler chicken. In addition, the Bacillus licheniformis-fermented product has the potential for development as feed additives and use as a possible solution to treat and/or prevent coccidiosis in the poultry industry. Further, Bacillus licheniformis-derived surfactin also exhibited the pathogen killing activity against Eimeria tenella. Therefore, the present invention indicates that the beneficial effects of Bacillus licheniformis-fermented products in broilers might alleviate coccidiosis.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows effects of surfactin standard and the Bacillus licheniformis-fermented product-derived surfactin C-form on the death of Eimeria tenella merozoites.

FIG. 2 shows sporulation of the Eimeria oocyst treated with surfactin standard and the Bacillus licheniformis-fermented product-derived surfactin C-form. Three experiments were conducted, and one representative result is displayed.

FIG. 3A shows scanning electron microscope images of Eimeria sporozoites illustrating the antiparasitic activity of different concentrations (a: 0 ppm; b: 5 ppm surfactin standard; c: 10 ppm surfactin standard; d: 20 ppm surfactin standard. Three experiments were conducted, and one representative result is presented.

FIG. 3B shows scanning electron microscope images of Eimeria sporozoites illustrating the antiparasitic activity of different concentrations (e: 5 ppm Bacillus licheniformis-fermented products-derived surfactin; f: 10 ppm Bacillus licheniformis-fermented products-derived surfactin; g: 20 ppm Bacillus licheniformis-fermented products-derived surfactin) of surfactin. Three experiments were conducted, and one representative result is presented.

FIG. 4 shows effect of Bacillus licheniformis-fermented products on oocyst per gram of feces (OPG) in broilers exposed to Eimeria tenella. NC=basal diet without treatment. PC=basal diet plus oral administration of Eimeria tenella. LBL=basal diet plus oral administration of Eimeria tenella and 1.25 g/kg of Bacillus licheniformis-fermented products. MBL=basal diet plus oral administration of Eimeria tenella and 2.5 g/kg of Bacillus licheniformis-fermented products. HBL=basal diet plus oral administration of Eimeria tenella and 5 g/kg of Bacillus licheniformis-fermented products. Values are expressed as mean (n=3).

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

According to the present invention, the medicament can be manufactured to a dosage form suitable for oral administration, using techniques well known to those skilled in the art, including, but not limited to, sterile powder, dispersible powder or granule, solution, suspension, and the like.

According to the present invention, the medicament may further comprise a pharmaceutically acceptable carrier which is widely used in pharmaceutically manufacturing techniques. For example, the pharmaceutically acceptable carrier can comprise one or more reagents selected from the group consisting of solvent, emulsifier, suspending agent, decomposer, disintegrating agent, dispersing agent, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar-containing solution, aqueous solution containing alcohol, and combinations thereof.

In addition, the composition according to the present invention can be added to chicken feed or drinking water using standard techniques well known to those skilled in the art. For example, the composition according to the present invention can be added directly to the chicken feed, or it can be used to produce one or more intermediate compositions (such as feed additives or premixes), which are then added to the chicken feed.

According to the present invention, the composition can be used as a food additive, added by the conventional method in the preparation of the raw material, or added during the preparation of food, and prepared with any edible material into food products for human and non-human animals.

According to the present invention, types of food products include, but not limited to, beverages, fermented foods, bakery products, health foods, dietary supplements, and feed additives.

### Example 1

Preparation of Bacillus licheniformis-Fermented Products

Bacillus licheniformis was purchased from the Biosource Collection and Research Center (BCRC 11556, Hsinchu, Taiwan). Solid-state fermentation substrates (1-5% glucose, 10-30% soybean meal, and 3-6% yeast) were mixed with water in a space bag to obtain 45-55% of initial moisture content, and the mixture was autoclaved at 121° C. for 30 min. The cooled substrates were inoculated with 4% (v/w) inoculum of Bacillus licheniformis, mixed carefully under sterile conditions, and incubated at 30° C. for 6 days in a chamber with free oxygen and relative humidity above 80%. Fermented products were dried at 50° C. for 2 days and homogenized through mechanical agitation. The fermented powder was then stored at 4° C. prior to analysis. For the determination of bacteria counts in fermented products, the fermented powder was diluted serially in 0.85% NaCl, plated on tryptic soy agar (Sigma-Aldrich, St. Louis, Mo.), and incubated for 18 h at 30° C. Bacterial growth was counted and is expressed as colony forming units per gram (CFU/g)(from 1.25×10⁶⁻⁷ CFU/g of feed to 5×10⁶⁻⁷ CFU/g of feed). For the determination of surfactin (Bacillus licheniformis-derived antibacterial cyclic lipopeptide) content in fermented products, the fermented powder was adjusted to pH 2.0 with concentrated HCl and incubated overnight at 4° C. The precipitate was dissolved in distilled water and extracted with methanol. The mixture was shaken vigorously, and the organic phase was concentrated at reduced pressure at 40° C. The extract was further filtered using a syringe filter with a 0.22-μm membrane. The surfactin concentration in the filtrate was measured using high-performance liquid chromatography.

### Example 2

Effect of surfactin and the Bacillus licheniformis-fermented product-derived surfactin C-form on treating and/or preventing coccidiosis in vitro

### 2.1 Statistical Analysis

Data were analyzed by one-way ANOVA using the GLM procedure of SAS software package (Version 9.4; SAS Institute, Cary, N.C.). Replicates were considered the experimental units. Results were expressed as mean±SD. Means were compared by employing Tukey's HSD test at a significance level of P<0.05.

### 2.2 In Vitro Anticoccidial Analysis

Intestinal samples from naturally infected broilers were obtained from poultry farms. Un-sporulated oocysts were obtained from the cecal contents of broilers at day 8 post-infection. Oocyst sporulation was conducted in an aqueous solution of 2% potassium dichromate (K₂Cr₂O₇) with or without surfactin standard or the Bacillus licheniformis-fermented product-derived surfactin C-form for 72 h at 25° C. (10⁴ oocysts/mL). Sporulated and non-sporulated oocysts were counted and the percentage of sporulation was estimated by counting the number of sporulated oocysts in a total of 100 oocysts by microscopic observation. Second-generation merozoites were collected from ceca at 110 h post-infection from chickens that had been inoculated with 1×10⁵ sporulated oocysts per bird. The merozoites (10⁴/mL) were treated with or without surfactin standard or the Bacillus licheniformis-fermented product-derived surfactin C-form for 1 h at 37° C. Three groups of merozoites were prepared, including: (1) a control group comprising 10⁴/ml merozoites without surfactin; (2) an experimental group comprising 10⁴/ml merozoites treated with 100 ppm surfactin standard; and (3) an experimental group comprising 10⁴/ml merozoites treated with the 100 ppm Bacillus licheniformis-fermented product-derived surfactin C-form. After incubation, propidium iodide was added to each well and then stained at 37° C. for 30 min in the dark. The stained samples were examined under a confocal laser-scanning microscope (ZEISS LSM800, Jena, Germany). The percentage of death of merozoite was determined by counting the number of dead merozoite in a total of 100 merozoites. The Eimeria sporozoites from vaccine were treated with different surfacitn for 1 h at 37° C. (10⁶ sporozoites/mL). The sporozoites were fixed with 2.5% glutaraldehyde in 50 mM potassium phosphate buffer (pH 6.8) overnight at 4° C., and then washed three times with 50 mM potassium phosphate buffer (pH 6.8). Dehydration was performed in different concentrations of ethanol (35-100%) for 10 mM each and the samples were dried in a critical point drier (CPD 030, Bal-Tec AG, Balzers, Liechtenstein). Specimens were coated under vacuum with gold:palladium (60:40) in a sputter coater (Bal-Tec AG, Balzers, Liechtenstein), and examined using a scanning electron microscope (JSM-6300, JEOL Ltd., Tokyo, JAPAN).

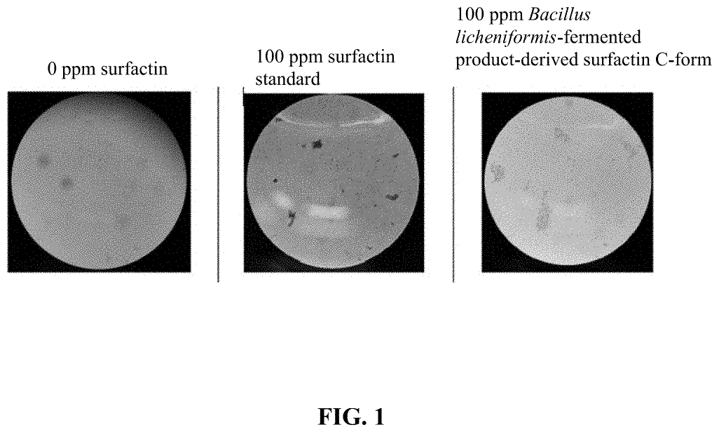

Results of confocal microscopy examinations showed that surfactin standard and the Bacillus licheniformis-fermented product-derived surfactin C-form are able to cause the death of merozoite of Eimeria tenella compared with control group (P<0.05, Table 1 and FIG. 1).

TABLE 1
Effect of surfactin standard and the Bacillus licheniformis-fermented
product-derived surfactin C-form on the death of merozoite in vitro

| | Death of merozoite (%)¹ |
|---|---|
| 0 ppm surfactin | 2 ± 0.03^{a} |
| 100 ppm surfactin standard | 97 ± 0.01^{b} |
| 100 ppm Bacillus licheniformis-fermented | 63 ± 0.01^{c} |
| product-derived surfactin C-form | |

¹Values are expressed as mean ± SD (n = 3).
^{a-c}Means within a column with no common superscript are significantly different (P < 0.05).

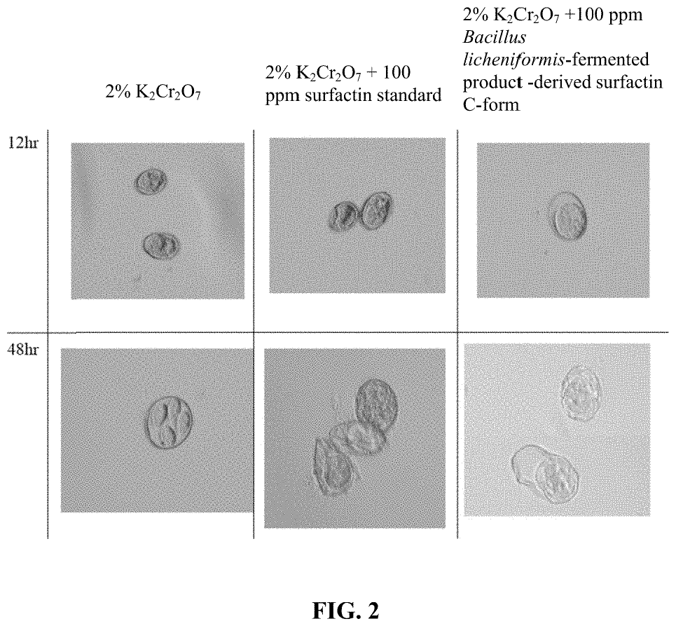

Furthermore, three groups of coccidia oocysts were prepared, including: (1) a control group comprising 10⁴/ml merozoites and 2% K₂Cr₂O₇ solution without surfactin; (2) an experimental group comprising 10⁴/ml merozoites and 2% K₂Cr₂O₇ solution treated with 100 ppm surfactin standard; and (3) an experimental group comprising 10⁴/ml merozoites and 2% K₂Cr₂O₇ solution treated with the 100 ppm Bacillus licheniformis-fermented product-derived surfactin C-form. The result indicated that the sporulation of the coccidia oocyst can be found after 48 hours treated with 2% K₂Cr₂O₇. However, surfactin standard and the Bacillus licheniformis-fermented product-derived surfactin C-form significantly inhibited the sporulation of the coccidia oocyst after 48 h treatment (P<0.05, Table 2 and FIG. 2).

TABLE 2
Effect of surfactin and the Bacillus licheniformis-
fermented product-derived surfactin
C-form on sporulation of the coccidia oocyst in vitro

| | Sporulation of the coccidia oocyst (%)¹ | | | |
|---|---|---|---|---|
| | 12 h | 24 h | 48 h | 72 h |
| 2% K₂Cr₂O₇ | 0 | 0 | 96 ± 0.03^{a} | 97 ± 0.02^{a} |
| 2% K₂Cr₂O₇ + | 0 | 0 | 0^{b} | 0^{b} |
| 100 ppm surfactin | | | | |
| 100 ppm Bacillus | 0 | 0 | 0^{b} | 0^{b} |
| licheniformis-fermented | | | | |
| product-derived surfactin | | | | |
| C-form | | | | |

¹Values are expressed as mean ± SD (n = 3).
^{a-b}Means within a column with no common superscript are significantly different (P < 0.05).

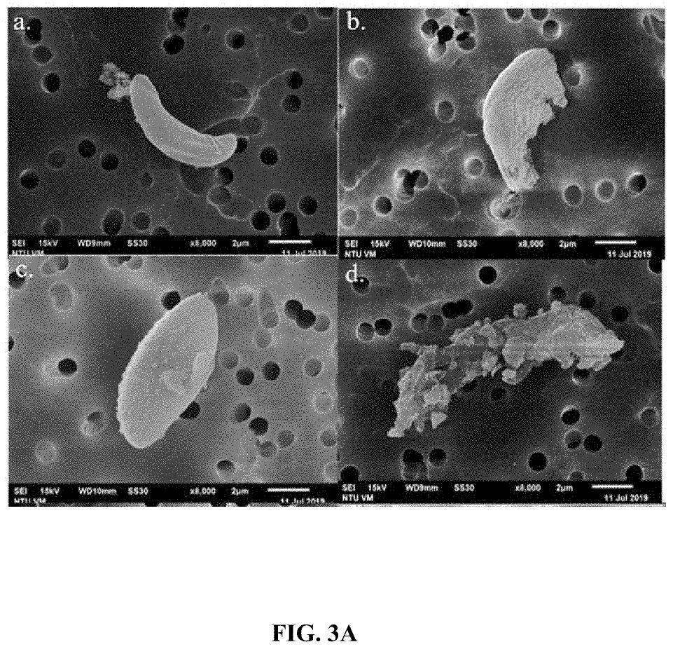

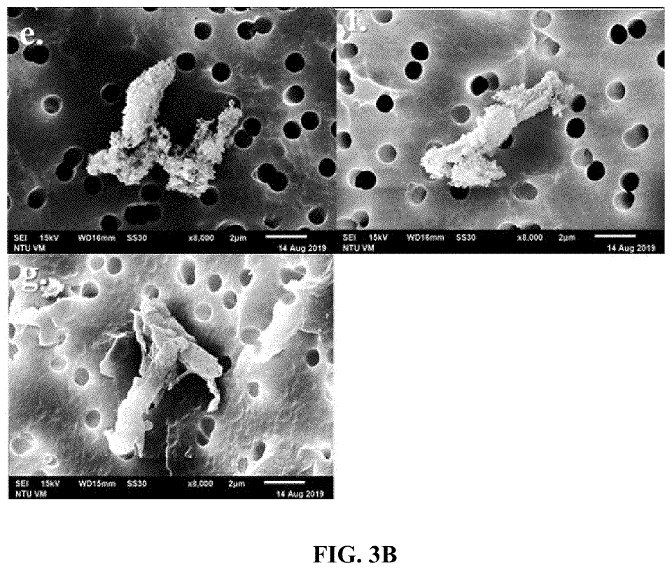

The results of scanning electron microscopy showed that surfactin standard could disrupt the morphology of Eimeria tenella sporozoites after 1 h treatment compared with the untreated group (FIG. 3A). Similarly, Bacillus licheniformis-fermented products-derived surfactin also damage the morphology of Eimeria tenella sporozoites (FIG. 3B).

### Example 3

Effect of Bacillus licheniformis-Fermented Product on Treating and/or Preventing Coccidiosis In Vivo

### 3.1 Statistical Analysis

Regarding statistical analysis, it can be referred to Example 2.1.

### 3.2 Animal Study

### 3.2.1 Anti-Coccidial Index, Cecal Lesion Score and Bloody Diarrhea Score

All experiments were performed in accordance with approved guidelines. The animal protocol was approved by the Institutional Animal Care and Use Committee of National Ilan University (IACUC, protocol number 107-12). One-day-old healthy male broiler chickens (Ross 308) were obtained from a local commercial hatchery. On day 1, 120 birds were randomly assigned to 5 treatments (with 6 replicates of 4 birds per cage) in a completely randomized design. Broilers were reared in stainless-steel, temperature-controlled cages (190 cm×50 cm×35 cm). The experimental diets consisted of (1) basal diet without treatment (NC), (2) basal diet plus oral administration of Eimeria tenella (PC), (3) basal diet plus oral administration of Eimeria tenella and 1.25 g/kg of Bacillus licheniformis-fermented products (1.25×10⁶⁻⁷ CFU/g of feed; 1.5-2.0 mg/g surfactin of feed) (LBL), (4) basal diet plus oral administration of Eimeria tenella and 2.5 g/kg of Bacillus licheniformis-fermented products (2.5×10⁶⁻⁷ CFU/g of feed; 3.5-4.0 mg/g surfactin of feed) (MBL), and (5) basal diet plus oral administration of Eimeria tenella and 5 g/kg of Bacillus licheniformis-fermented products (5×10⁶⁻⁷ CFU/g of feed; 7.0-8.0 mg/g surfactin of feed) (HBL). The diets were formulated to meet or exceed the requirements of birds according to the National Research Council recommendations (NRC, Nutrient Requirements for Poultry, 1994, Table 3). Feed and water were available ad libitum throughout the experiment. The temperature was set at 32° C. on the first day, gradually reduced to 24° C. by the third week, and then maintained at 24° C. to the end of the experiment. The lighting schedule was 22L: 2D throughout the experiment. Broilers were vaccinated by nose drop administration with combined Newcastle disease-infectious bronchitis vaccines on days 4 and 14. The birds were orally inoculated with Eimeria tenella oocyst (5×10⁴) on 14 d of age. Their average body weight, average daily gain, average daily feed intake, and feed conversion ratio were calculated from days 1 to 28.

TABLE 3
Composition of basal diets

| Item | 1 to 28 d |
|---|---|
| Ingredient, g kg⁻¹ | |
| Corn, yellow | 511.8 |
| Soybean meal, 36.7% CP | 350.0 |
| Fish meal | 100.0 |
| CaCO₃, 38% | 20.0 |
| CaHPO₄ | 10.0 |
| DL-Methionine, 99.5% | 2.0 |
| Mineral premix¹ | 1.0 |
| Vitamin premix² | 1.0 |
| Choline chloride, 50% | 0.2 |
| Sodium chloride | 4.0 |
| Calculated value, g kg⁻¹ | |
| Crude protein | 230.0 |
| Analyzed calcium | 13.8 |
| Analyzed total phosphorus | 7.0 |
| Lysine | 13.4 |
| Methionine + Cystine | 10.0 |
| ME, kcal/kg | 3411 |

¹Supplied per kilogram of diet: Cu (CuSO₄•5H₂O), 20 mg; Zn (ZnO), 100 mg; Fe (FeSO₄•H₂O), 140 mg; Mn (MnSO₄•H₂O), 4 mg; Se (Na₂SeO₃), 0.1 mg and I (ethylenediamine dihydriodide), 0.2 mg.
²Supplied per kilogram of diet: vitamin A, 6,000 IU; vitamin D3, 900 IU; vitamin E, 30 IU; vitamin K₃, 3 mg; riboflavin, 6 mg; niacin, 60 mg; pantothenic acid, 18 mg; and vitamin B₁₂, 30 μg.

### 3.3 Measurement of Anti-Coccidial Index

The extent of bloody diarrhea was assigned to one of 3 degrees (from + to +++) according to the mean pieces of bloody feces in each group. Briefly, + represents normal feces; ++ stands for one to two pieces; +++, three or more pieces of bloody feces, respectively. All birds in each cage were weighed weekly and relative body weight gain was calculated. Survival rate and bird appearance were checked daily. Lesion scores in the cecum were scored based on a scale of 0 to 4, wherein 0 is normal and 1 to 4 indicate increasing severity of the infection. 0=normal tissue without gross lesions (0% blood in feces); 1=few scattered petechiae on gut wall (25% blood in feces); 2=numerous petechiae (50% blood in feces); 3=extensive hemorrhage (75% blood in feces); 4=extensive hemorrhage with dark color in guts (>75% blood in feces). The oocyst per gram of feces (OPG) was obtained by the collection of feces from broilers at the indicated time points. 100 fresh feces from each group were collected, weighed and suspended in water. The oocysts were suspended in saturated salt water and counted on two McMaster chambers after filtration and centrifugation. The average oocyst number in the feces of each group was obtained. In addition, the feces from each group were classified into normal, soft and bloody types and scored at the indicated time points. On 28 d of age, Anti-coccidial index (ACI) was calculated based on the following formula, ACI=[relative body weight gain (RBWG, %)+survival rate (SR, %)]−[lesion score index (LSI)+oocyst count index (OI)].

3.4 Results After Eimeria tenella challenge, cecal lesion score in broilers was significantly increased compared with control group (P<0.05, Table 4). However, the dietary supplementation of 5 g/kg of Bacillus licheniformis-fermented products in broilers significantly reduced the cecal lesion score in broilers exposed to Eimeria tenella (P<0.05, Table 5). Similarly, the bloody diarrhea score was elevated in broilers under Eimeria tenella challenge, whereas dietary supplementation of Bacillus licheniformis-fermented products (1.25, 2.5 and 5 g/kg) was able to reduce the bloody diarrhea score in broilers (Table 5). Furthermore, the bloody diarrhea score in 5 g/kg of Bacillus licheniformis-fermented products-fed broilers under Eimeria tenella challenge was recover to normal status compared with other groups (Table 4).

TABLE 4
Effect of Bacillus licheniformis-fermented products
on cecal lesion score and bloody diarrhea
score in broilers exposed to Eimeria tenella.

| | Cecal | Bloody |
|---|---|---|
| | lesion score⁶ | diarrhea score |
| NC¹ | 1.11 ± 0.78^{b} | + |
| PC² | 3.56 ± 0.53^{a} | +++ |
| LBL³ | 2.89 ± 0.78^{a} | ++ |
| MBL⁴ | 2.78 ± 0.67^{a} | ++ |
| HBL⁵ | 1.67 ± 0.71^{b} | + |

¹NC = basal diet without treatment.
²PC = basal diet plus oral administration of Eimeria tenella.
³LBL = basal diet plus oral administration of Eimeria tenella and 1.25 g/kg of Bacillus licheniformis-fermented products.
⁴MBL = basal diet plus oral administration of Eimeria tenella and 2.5 g/kg of Bacillus licheniformis-fermented products.
⁵HBL = basal diet plus oral administration of Eimeria tenella and 5 g/kg of Bacillus licheniformis-fermented products.
⁶Values are expressed as mean ± SD (n = 3).
^{a-b}Means within a column with no common superscript are significantly different (P < 0.05).

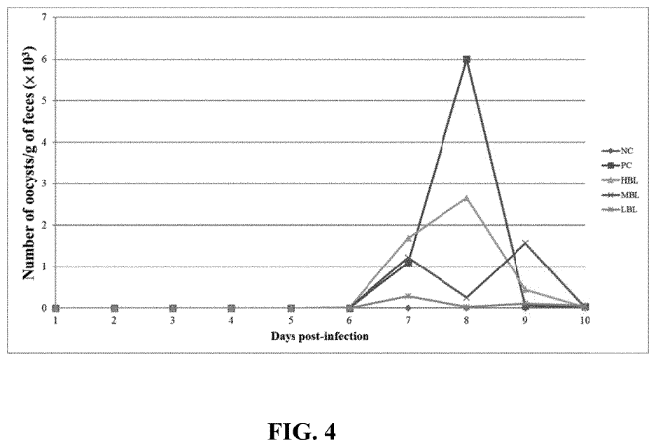

The number of oocyst per gram of feces in broilers was increased after Eimeria tenella infection, while Bacillus licheniformis-fermented products could reduce the number of oocyst per gram of feces in broilers under Eimeria tenella challenge (FIG. 4). The survival rate was decreased in broilers exposed to Eimeria tenella compared with control group, whereas dietary supplementation of Bacillus licheniformis-fermented products (1.25, 2.5 and 5 g/kg) in broilers could increase the survival rate (Table 5). The oocyst count index in the feces was increased in broilers under Eimeria tenella challenge with other groups (Table 5). Dietary supplementation of Bacillus licheniformis-fermented products increases the oocyst count index in the feces of broilers exposed to Eimeria tenella in a dose dependent manner (Table 5). The lesion score index was increased in broilers under Eimeria tenella challenge compared with control group, whereas dietary supplementation of Bacillus licheniformis-fermented products (1.25, 2.5 and 5 g/kg) in broilers could decrease the lesion score index (Table 5). The Eimeria tenella challenge reduced the anti-coccidial index in broilers, whereas dietary supplementation of Bacillus licheniformis-fermented products could reverse the anti-coccidial index in broilers exposed to Eimeria tenella (Table 5).

TABLE 5
Effect of Bacillus licheniformis-fermented products on the
anti-coccidial index of broilers exposed to Eimeria tenella.

| | RBWG (%)⁶ | SR (%)⁷ | OI (%)⁸ | LSI (%)⁹ | ACI¹⁰ |
|---|---|---|---|---|---|
| NC¹ | 100.0 | 100.0 | 0.0 | 11.1 | 188.9 |
| PC² | 92.9 | 88.9 | 40.0 | 35.6 | 106.3 |
| LBL³ | 97.9 | 100.0 | 2.7 | 28.9 | 166.3 |
| MBL⁴ | 106.0 | 100.0 | 17.1 | 27.8 | 161.1 |
| HBL⁵ | 96.1 | 100.0 | 26.8 | 16.7 | 152.6 |

¹NC = basal diet without treatment.
²PC = basal diet plus oral administration of Eimeria tenella.
³LBL = basal diet plus oral administration of Eimeria tenella and 1.25 g/kg of B. licheniformis-fermented products.
⁴MBL = basal diet plus oral administration of Eimeria tenella and 2.5 g/kg of Bacillus licheniformis-fermented products.
5HBL = basal diet plus oral administration of Eimeria tenella and 5 g/kg of Bacillus licheniformis-fermented products.
⁶RBWG = relative body weight gain. RBWG = (100 × body weight gain per group)/[body weight gain of the NC group].
⁷SR = survival rate. SR = (100 × the number of living chickens)/(total number of chickens per group).
⁸OI = oocyst count index. OI = 100 × 0.4 × (oocyst counts per group)/[oocyst counts for PC group].
⁹LSI = lesion score index. LSI = 10 × (lesion score per group).
¹⁰ACI = anti-coccidial index. ACI = [RBWG (%) + SR (%)] − [LSI (%) + OI (%)]. ACI ≥ 160: sensitive to the anti-coccidial drug; 160 > ACI ≥ 120: partially resistant to the anti-coccidial drug; ACI < 120: resistant to the anti-coccidial drug.

Surfactin is a secondary metabolite produced from Bacillus species and it consists of multiple isoforms (Haddad N I, Liu X, Yang S, Mu B. 2008. Surfactin isoforms from Bacillus subtilis HSO121: separation and characterization. Protein Pept. Lett. 15:265-269; Sousa M, Dantas I T, Feitosa F X, Alencar A E V, Soares S A, Melo V M M, Gonsalves L R B, Sant'ana H B. 2014. Performance of a biosurfactant produced by Bacillus subtilis LAMI005 on the formation of oil/biosurfactant/water emulsion: study of the phase behaviour of emulsified systems. Braz. J. Chem. Eng. 31:613-623. doi: 10.1590/0104-6632.20140313s00002766; Sumi C D, Yang B W, Yeo I C, Hahm Y T. 2015. Antimicrobial peptides of the genus Bacillus: a new era for antibiotics. Can. J. Microbiol. 61:93-103. doi: 10.1139/cjm-2014-0613). Several factors, such as environmental and nutritional conditions, determine the surfactin isoforms (Peypoux F, Michel G 1992. Controlled biosynthesis of Va17- and Leu7-surfactins. Appl. Microbiol. Biotechnol. 36:515-517. doi:10.1007/BF00170194; Kowall M, Vater J, Kluge B, Stein T, Franke P, Ziessow D. 1998. Separation and characterization of surfactin isoforms produced by Bacillus subtilis OKB 105. J. Colloid Interface Sci. 204:1-8). The structure of surfactin includes seven amino acid peptide loop and a hydrophobic fatty acid chain (Kowall M, Vater J, Kluge B, Stein T, Franke P, Ziessow D. 1998. Separation and characterization of surfactin isoforms produced by Bacillus subtilis OKB 105. J. Colloid Interface Sci. 204:1-8). Among Bacillus species, Bacillus licheniformis also has the ability to synthesize surfactin (Pecci Y, Rivardo F, Martinotti MG, Allegrone G 2010. LC/ESI-MS/MS characterisation of lipopeptide biosurfactants produced by the Bacillus licheniformis V9T14 strain. J. Mass Spectrom. 45:772-778; Sumi C D, Yang B W, Yeo I C, Hahm Y T. 2015. Antimicrobial peptides of the genus Bacillus: a new era for antibiotics. Can. J. Microbiol. 61:93-103. doi: 10.1139/cjm-2014-0613). Surfactin shows antibacterial activity against a wide range of Gram-positive bacteria, such as Listeria monocytogenes and Methicillin-resistant S. aureus, but does not cause hemolysis or inhibit the growth of Gram-negative bacteria (Dischinger J, Josten M, Szekat C, Sahl H G, Bierbaum G 2009. Production of the novel two-peptide lantibiotic lichenicidin by Bacillus licheniformis DSM 13. PLoS One 4:e6788. doi:10.1371/journal.pone.0006788; Abdel-Mohsein H, Sasaki T, Tada C, Nakai Y. 2011. Characterization and partial purification of a bacteriocin-like substance produced by thermophilic Bacillus licheniformis H1 isolated from cow manure compost. Anim Sci. J. 82:340-351. doi:10.1111/j.1740-0929.2010.00835.x). Prior arts have demonstrated that Bacillus licheniformis-fermented product-derived surfactin C-form can cause the death of Brachyspira hyodysenteriae and C. perfringens and inhibit the growth of B. hyodysenteriae and C. perfringens in vitro (Horng Y B, Yu Y H, Dybus A, Hsiao F S H, Cheng Y H. 2019. Antibacterial activity of Bacillus species-derived surfactin on Brachyspira hyodysenteriae and Clostridium perfringens. AMB Express 9:188. doi: 10.1186/s13568-019-0914-2). In addition to pathogens, it has been reported that surfactin exhibits antiparasitic activity against Nosema ceranae and Plasmodium falciparum (Chakrabarty S P, Saikumari Y K, Bopanna M P, Balaram H. 2008. Biochemical characterization of Plasmodium falciparum Sir2, a NAD+-dependent deacetylase. Mol. Biochem. Parasitol. 158:139-151. doi: 10.1016/j.molbiopara.2007.12.003; Porrini M P, Audisio M C, Sabaté D C, Ibarguren C, Medici S K, Sarlo E G, Garrido P M, Eguaras M J. 2010. Effect of bacterial metabolites on microsporidian Nosema ceranae and on its host Apis mellifera. Parasitol. Res. 107:381-388. doi: 10.1007/s00436-010-1875-1). Surfactin is a potent inhibitor of intraerythrocytic growth of P. falciparum through inhibition of NAD and acetylated peptide (Chakrabarty S P, Saikumari Y K, Bopanna M P, Balaram H. 2008. Biochemical characterization of Plasmodium falciparum Sir2, a NAD+-dependent deacetylase. Mol. Biochem. Parasitol. 158:139-151. doi: 10.1016/j.molbiopara.2007.12.003). Here, the applicant further demonstrated that surfactin not only inhibited the sporulation of the Eimeria tenella oocyst but also promoted the death of Eimeria tenella merozoite and disrupted the morphology of sporozoites in vitro. The precise mechanism of how surfactin interacts with oocyst, merozoite, and sporozoite to exert antiparasitic activity remains to be investigated. Taken together, Bacillus licheniformis-fermented product-derived surfactin C-form not only exhibits antibacterial activity, it also has antiparasitic activity in vitro.

Coccidiosis caused by Eimeria species represents a devastating impact on the poultry industry due to high morbidity, mortality and significant costs resulting from prophylaxis and treatment. The life cycle of Eimeria begins when sporulated oocysts are picked up and swallowed by the chickens. The sporozoites are released from oocysts after grinding in the gizzard and enzymatic digestion in the gut. The sporozoites imbed in the intestinal lining and develop into merizoites, followed by multiplying several times and damaging tissue of chicken (Price K R, Freeman M, Van-Heerden K, Barta J R. 2015. Shedding of live Eimeria vaccine progeny is delayed in chicks with delayed access to feed after vaccination. Vet. Parasitol. 208:242-245. doi: 10.1016/j.vetpar.2015.01.009). The life cycle of Eimeria takes about four to seven days to complete. Because of the complexity of life cycle of Eimeria, controlling avian coccidiosis is a challenge in the poultry industry. Further, the ban on the use of antibiotics in animal feed and overuse of drugs for coccidiosis in poultry is also a critical factor affecting the prevention of coccidiosis. Recently, probiotics have been considered as alternative candidates for the prevention of coccidiosis in poultry. In the present invention, the applicant further demonstrated that 5 g/kg of Bacillus licheniformis-fermented products in broilers reduced the cecal lesion score and bloody diarrhea score in broilers exposed to Eimeria tenella. Dietary supplementation of Bacillus licheniformis-fermented products elevated the anti-coccidial index in broilers under Eimeria tenella challenge. These findings demonstrated that Bacillus licheniformis-fermented products could normalize Eimeria tenella-induced negative impact on broilers.

In conclusion, Bacillus licheniformis-fermented product-derived surfactin C-form and Bacillus licheniformis-fermented products exhibit anti-coccidial activity in vitro and in vivo. Surfactin can interfere with the life cycle of Eimeria tenella and the anti-coccidial action has an advantage over chemical anti-coccidial drugs. Bacillus licheniformis-fermented products not only contain live microorganisms but also have Bacillus licheniformis-derived surfactin. This invention demonstrates the feasibility of Bacillus licheniformis-fermented products as a veterinary medicine for controlling coccidiosis in broilers.

Therefore, the composition comprising an effective amount of a Bacillus licheniformis-fermented product provides the effect on treating and/or preventing coccidiosis through inhibiting sporulation of oocysts from the Eimeria tenella, promoting death of merozoite from the Eimeria tenella, disrupting morphology of sporozoites from the Eimeria tenella, and alleviating cecal lesion and bloody diarrhea in the broiler chicken. In addition, the Bacillus licheniformis-fermented product has the potential for development as feed additives and use as a possible solution to treat and/or prevent coccidiosis in the poultry industry. Further, Bacillus licheniformis-derived surfactin also exhibited the pathogen killing activity against Eimeria tenella. Therefore, the present invention indicates that the beneficial effects of Bacillus licheniformis-fermented products in broilers might alleviate coccidiosis.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for treating and/or preventing coccidiosis, comprising administering to a subject in need thereof a composition comprising an effective amount of a Bacillus spp.-fermented product.

2. The method according to claim 1, wherein the Bacillus spp. is Bacillus licheniformis.

3. The method according to claim 2, wherein the effective amount of the Bacillus licheniformis-fermented product is at least 1.25 g/kg.

4. The method according to claim 2, wherein the coccidiosis is induced by Eimeria spp.

5. The method according to claim 4, wherein the coccidiosis is induced by Eimeria tenella.

6. The method according to claim 5, wherein the coccidiosis is treated and/or prevented by inhibiting sporulation of oocysts from the Eimeria tenella.

7. The method according to claim 5, wherein the coccidiosis is treated and/or prevented by promoting death of merozoite from the Eimeria tenella.

8. The method according to claim 5, wherein the coccidiosis is treated and/or prevented by disrupting morphology of sporozoites from the Eimeria tenella.

9. The method according to claim 2, wherein the subject in need thereof is a poultry.

10. The method according to claim 9, wherein the poultry is a broiler chicken.

11. The method according to claim 10, wherein the coccidiosis is treated and/or prevented by alleviating cecal lesion and bloody diarrhea in the broiler chicken.

12. The method according to claim 2, wherein the Bacillus licheniformis-fermented product comprises at least one antibacterial cyclic lipopeptide.

13. The method according to claim 12, wherein the at least one antibacterial cyclic lipopeptide is surfactin.

14. The method according to claim 13, wherein the surfactin is at least in an amount of 5 ppm.

15. The method according to claim 2, wherein the composition further comprises a pharmaceutically acceptable carrier.

16. The method according to claim 2, wherein the composition is in a form of powder, granule, liquid, gel or paste.

17. The method according to claim 2, wherein the composition is prepared in a form of a medicament or a food product.

18. The method according to claim 17, wherein the food product is a feed additive.

19. The method according to claim 17, wherein the medicament is in a dosage form for oral administration.

20. The method according to claim 19, wherein the dosage form is selected from the group consisting of solution, suspension, and powder.
